(19) United States
(12) Patent Application Publication
(10) Pub. No.: US 2002/0110601 A1
(43) Pub. Date: Aug. 15, 2002

(54) Antineoplastic platinum therapeutic method and composition

(76) Inventors: Roman Perez-Soler, Setauket, NY (US); Dong Shin, Pittsburgh, PA (US); Garrett Walsh, Houston, TX (US)

Correspondence Address: Thomas M. Saunders, Lorusso & Loud, 440 Commercial Street, Boston, MA, 02109, US

(21) Appl. No.: 09/841,480

(22) Filed: Apr. 24, 2001

Related U.S. Application Data
(60) Provisional application No. 60/200,218, filed on Apr. 28, 2000.
(60) Provisional application No. 60/193,565, filed on Mar. 31, 2000.

(51) Int. Cl. A61K 33/24; A61K 31/555; A61K 31/28

(52) U.S. Cl. 424/649; 514/184; 514/492

(57) ABSTRACT

A sepsis-avoiding and hydrostatic-neutral method of administering platinum therapeutic agents into a body cavity of a subject by means of introduction through a hydrostatic-tight indwelling catheter.

### STATEMENT OF GOVERNMENT INTERESTS

The U.S. Government has rights in this invention pursuant to Grant No. CA 60496 awarded by the National Institutes of Health.

### FIELD OF THE INVENTION

A sepsis-avoiding and hydrostatic-neutral method of administering platinum therapeutic agents into a body cavity of a subject by means of introduction through an indwelling catheter.

### BACKGROUND OF THE INVENTION

Certain neoplastic diseases including cancers, with particular reference to malignant mesothelioma, have drawn considerable attention from clinicians, epidemiologists, pathologists and even the lay public because of a conspicuous association with the environmental carcinogens, notably asbestos. When linked to asbestos exposure, malignant mesothelioma appears only after a long latency interval-often 30 or more years. Mesothelioma characteristically develops in men 50 to 80 years old (median, 60 years). Approximately 60% arise on the right, and fewer than 5% of patients present with bilateral involvement. Locally aggressive invasion into the lung, pericardium, and heart often results in end stage of the disease and is fatal, although greater than 30% of cases in autopsy series have distant metastasises.

Surgery had a very limited role in therapy in the past, although exploratory thoracotomy has often been necessary to obtain adequate tissue to provide accurate histologic diagnosis. Aggressive surgical procedures yield relatively high rates of morbidity, with two-year survival rates of 10% to 37% and five-year survival rates of 10% or less, leading many investigators to suggest that an aggressive surgical approach that included extrapleural pneumonectomy did not prolong the overall survival of patients. Studies of the potential utility of radiotherapy for malignant mesothelioma have shown that radiotherapy alone does not prolong survival in this disease. The median duration of survival in these studies is about 10 months, which is not significantly different from the natural history of the disease. Radiotherapy, however, remains an important modality in controlling pain and other palliative measures.

Without being bound by any particular theory, it appears that in malignant pleural mesothelioma, the pleural space is the site of origin of the disease and it is by local invasion from the pleural space that the disease eventually causes the death of the patient. Therefore, approaches to improve local control of the disease by increasing the local drug concentration and the time of exposure of the tumor to the drug have an important impact on the natural course of the disease.

The antineoplastic drug NDDP is a lipophilic cisplatin analog useful in the practice of the present invention. It is particularly useful in liposomal form (L-NDDP). The relatively large size of the liposomes used most frequently with NDDP, in therapeutic uses, when administered i.v., accumulate in liver and spleen. Such formulation has limited ability to target solid tumors after i.v. administration. Clearly, an alternative mode of drug delivery is required.

### BRIEF DESCRIPTION OF THE DRAWINGS

FIG. 1 is a diagrammatic view of a catheter from outside the patient with the catheter as implanted in an intercostal space and into the pleural wall.

FIG. 2 is a diagrammatic skeletal view of the catheter at the site of placement.

### SUMMARY OF THE INVENTION

This invention comprises a sepsis-avoiding method of administering platinum therapeutic agents into a body cavity of a subject by means of introduction through an indwelling catheter. Noted are NDDP, well as cisplatin (and derivatives such as polyamidoamine (PAMAM) dendrimer generation 3.5 with a sodium carboxylate surface conjugated to cisplatin giving a dendrimer-platinate (dendrimer-Pt; 20-25 wt % platinum), nedaplatin, JM335 (trans-ammine (cyclohexylaminedichlorodihydroxo) platinum(IV)), and its platinum(II) dichloro homolog JM334 (including their cis isomeric counterparts (JM149 for JM335 and JM118 for JM334), also JM216 as well as ZD0473 (cis-amminedichloro(2-methylpyridine) platinum(II)); carboplatin, oxaliplatin, iproplatin; the dinuclear platinum complexes, BBR3005 ([trans-PtCl(NH3)22H2N(CH2)6NH2]2+), BBR3171 ([cis-PtCl(NH3)22H2N(CH2)6NH2]2+) and the trinuclear platinum complex, BBR3464 ([trans-PtCl(NH3)22 mu-trans-Pt(NH3)2(H2N(CH2)6NH2)2]4+); sterically hindered platinum complex, AMD473 [cis-aminedichloro(2-methylpyridine) platinum (II)]. In some embodiments, the method comprises administering platinum therapeutic agents into a body cavity of a subject by the step of hydrostatic-neutral introduction. Such agents are usefully administered in liposomal form, with particular reference to L-NDDP.

### DETAILED DESCRIPTION OF THE INVENTION

This invention is best understood with reference to the following definitions.

A. Platinum therapeutic agent is a term broadly understood to mean L-NDDP, well as cisplatin (and derivatives such as polyamidoamine (PAMAM) dendrimer generation 3.5 with a sodium carboxylate surface conjugated to cisplatin giving a dendrimer-platinate (dendrimer-Pt; 20-25 wt % platinum), nedaplatin, JM335 (trans-ammine (cyclohexylaminedichlorodihydroxo) platinum(IV)), and its platinum(l) dichloro homolog JM334 (including their cis isomeric counterparts (JM1 49 for JM335 and JM118 for JM334), also JM216 as well as ZD0473 (cis-amminedichloro(2-methylpyridine) platinum(II)); carboplatin, oxaliplatin, iproplatin; the dinuclear platinum complexes, BBR3005 ([trans-PtCl(NH3)22H2N(CH2)6NH2]2 +), BBR3171 ([cis-PtCl(NH3)22H2N(CH2)6NH2]2 +) and the trinuclear platinum complex, BBR3464 ([trans-PtCl(NH3)22 mu-trans-Pt(NH3)2(H2N(CH2)6NH2)2]4+); sterically hindered platinum complex, AMD473 [cis-aminedichloro(2-methylpyridine) platinum (II)],

B. NDDP is a lipophilic cisplatin analog designed and synthesized to be delivered in a liposomal carrier for the treatment of malignant diseases whose biology or location make them particularly accessible or sensitive to cytotoxic agents entrapped in liposomes. The structure of NDDP includes two branched aliphatic leaving groups containing ten carbon atoms and the cyclohexane group attached to the amino groups. The structure of NDDP is more fully set forth in U.S. Pat. No. 5,843,475 to Perez-Soler et al., the teachings of which are incorporated herein by reference.

C. L-NDDP shall mean NDDP entrapped in liposomes with particular reference to multilamellar liposomes. In some embodiments, the liposomes are composed of dimyristoylphosphatidyl choline (DMPC) and dimyristoylphosphatidyl glycerol (DMPG). Reference is made to DMPC:DMPG at a 7:3 molar ratio. Embodiments of L-NDDP are more fully set forth in U.S. Pat. No. 5,843,475 to Perez-Soler et al. Without being bound by any particular theory, it is believed that L-NDDP of multilamellar liposomes of DMPC:DMPG at a 7:3 molar ratio offers favorable NDDP entrapment biological profile. L-NDDP was found to be superior to cisplatin in that it was not cross-resistant in vitro or in vivo against a human cell line (LoVo carcinoma) and L1210/PDD leikemia, respectively. It was also more active than cisplatin against liver metastasises of M5076 reticulosarcoma by the i.v. route. L-NDDP, unlike cisplatin, is also not nephrotoxic in mice and dogs. In preclinical pharmacology studies, blood, liver and spleen levels of elemental Pt after the administration of L-NDDP were several fold higher than those achieved after the administration of an equimolar dose of cisplatin.

Reference is made to Perez-Soler R. et al., “Phase I clinical and pharmacology study of liposome-entrapped cis-bis-neodecanoato-trans-R,R-1,2-diaminocyclohexane-platinum (II).” Cancer Res. 50:4254-4259, 1990. Enhanced transcellular transport of L-NDDP is noted. As provided under the present invention L-NDDP displays an enhanced ability to penetrate into the tumor tissue from the pleural cavity. Particular note is made of liposomes of size ranging between one and three μm.

D. Body cavities, as related to cancer therapy, shall include the peritoneal cavity (as with ovarian carcinoma) and the pleural cavity (as with mesothelioma).

E. Indwelling catheters refers to biocompatible catheter devices which permit granulation tissue to amass around the catheters at the point of entry, and a step in wound healing. The granulation tissue then seals the point of catheter entry to prevent extravasation of platinum therapeutic agent. This state is termed a hydrostatic-tight indwelling catheter.

Any of a variety of suitable catheters are know in the art. By way of example, the silicone rubber Tenckhoff catheters (Bard Access, Salt Lake City) and Denver catheters are useful in the practice of this invention.

F. Sepsis-avoiding extravasation-free refers to, respectively, the substantial absence of platinum drug leakage at the site of drug introduction. This means the avoidance extravasation of administered drug. Sepsis is the clinical condition in which infective agents (e.g., bacteria, pathogenic fungi) or products of infection (e.g., bacterial toxins) enter the blood circulation or create a necrotic area at the point of catheter insertion which profoundly affect a patient's blood pressure, heart rate, and body temperature. Extravasation refers to at least about a 10 ml release of a liquid dosage administered through an indwelling catheter exiting at the site of introduction. Extravasation avoidance means such losses are minimized as is exudation and inflamation. This further presumes substantial cavity integrity.

G. Hydrostatic-neutral refers to introduction of a suspended platinum therapeutic agent into a fluid containing body cavity, such as the pleural cavity, without raising the hydrostatic pressure. This is accomplished in a number of ways. In cases of mesothelioma, in some embodiments, pleural fluid is removed just before or during the time the platinum therapeutic agent of about equal or lesser volume is introduced. This method would be volume-neutral. However, drug therapy is also usefully accompanied by lung deflation. Lung deflation or collapse leaves a void in the pleural space which permits introduction of a platinum drug solution without accompanying increase in hydrostatic pressure. The lung may be subsequently reinflated. Hydrostatic-neutral means no more than about a 20% increase in intrapleural pressure accompanies drug introduction and preferably less than about 10% and more particularly less than about 5%.

Caution must be exercised to be sure that there is no adhesion or pocket within the intrapleural space at the site of drug introduction to cause a local but substantial increase in hydrostatic pressure at that site.

Intrapleural L-NDDP has now been tested establishing the efficacy of this agent in malignant pleural mesothelioma. It has been discovered that prolonged and direct exposure of mesothelioma to the maximum tolerated dose of a lipophilic cylotoxic agent with enhanced tumor penetration properties translates into a significant therapeutic advantage compared with i.v. chemotherapy.

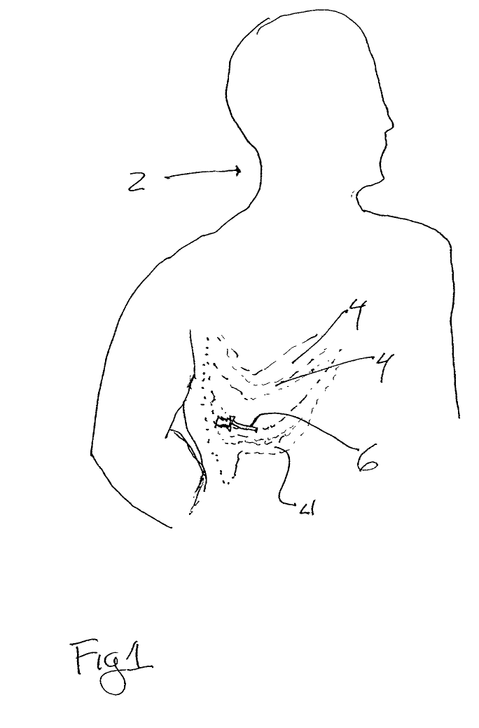

FIG. 1 is a diagrammatic view of a catheter from outside the patient with the catheter as implanted in an intercostal space and into the pleural wall. A human subject (2) with ribs shown in phantom (4) has a catheter (6) inserted between ribs and entering the pleural cavity.

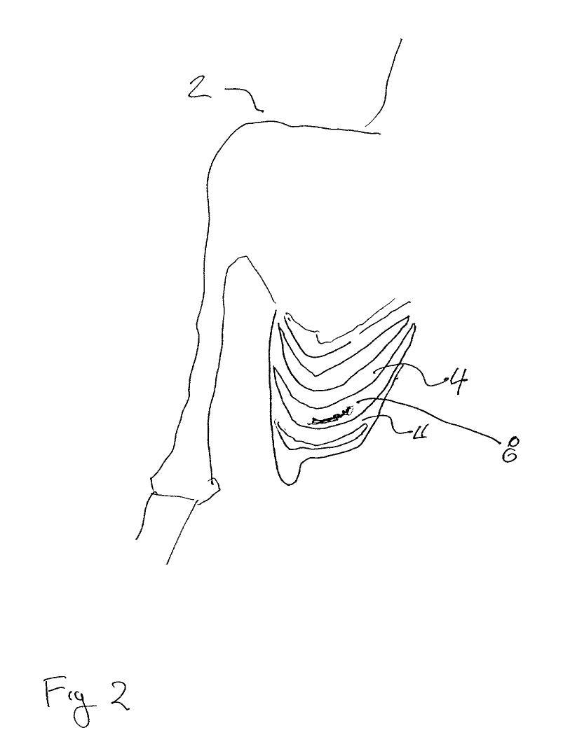

FIG. 2 is a diagrammatic skeletal view of a human subject (2) and ribs (4) with the catheter (6) as seen residing within the pleural space (8).

A Phase I trial using the procedures of the present invention employed L-NDDP administered by intrapleural administration in patients with malignant pleural effusions. No systemic toxicity was observed at doses that are two-fold higher than the MTD by the i.v. route, thus indicating an advantageous depot effect. A total of 21 patients were entered in this protocol. No re-accumulation of malignant pleural fluid over prolonged periods of time was observed in 9 of 19 evaluable patients. Negative cytology without fluid reaccumulation was observed in three patients. Four of five patients with mesothelioma showed response or tumor control.

Large multilamellar liposomal vesicles (median range 1-3 μm) introduced into body cavities are retained for a long period of time inside the cavity. Intracavitary administration of cytotoxic agents entrapped in mutilamellar vesicles results in higher and more prolonged intracavitary drug levels when compared with free drug administration. Slow L-NDDP absorption from the pleural cavity into the systemic circulation enhances the cytotoxic direct contact effect against tumor tissue open to the cavity.

It is noted that in some embodiments, cisplatin and L-NDDP administration are distinct. The transmembrane transport system of L-NDDP is qualitatively and quantitatively different from that of cisplatin. The cellular uptake of L-NDDP in tumor cells, both sensitive and resistant to cisplatin, is several fold higher than that of cisplatin. Without being bound by any particular theory, it is believed that because of its fast diffusion across membranes, L-NDDP is associated with enhanced ability to penetrate into the deeper tumor layers that are not in direct contact with a cavity into which drug is introduced according to the present method. The Phase I clinical study of L-NDDP by intrapleural administration in patients with malignant pleural effusions supports this belief.

Therapy began with a starting dose was 75 mg/m². Subsequent dose escalations were 1 50, 300, 375, 450, and 550 mg/m². Four patients were treated at the highest tested dose of 550 mg/m². None of the patients developed myelosuppression which is the dose limiting toxicity of L-NDDP at the MTD (300 mg/m²). Toxicities in these four patients were nausea and vomiting (grade 2) in all four patients and local pain in three of them (severe in one of them) probably secondary to chemical pleuritis. At lower dose levels, no local pain was observed and the only toxicity was mild nausea and vomiting. It is important to note that patients did not receive antiemetic premedication. Decreased reaccumulation of pleural fluid with negative cytology was observed in three patients. Loculation of fluid with no reaccumulation was observed in nine patients, in two patients lasting more than a year. Reaccumulation of fluid was observed in seven patients. Four of five patients with mesothelioma responded to the treatment.

In one embodiment of the present invention, L-NDDP preparation is usefully performed as described in Perez-Soler R, K Francis, S Al-Baker, et al., “Preparation and characterization of a liposomal preparation containing a lipophilic cisplatin derivative for clinical use,” J Microencapsulation 11:41-54, 1994. L-NDDP is conveniently prepared as a lyophilized powder in bottles containing 100 mg NDDP and 1,500 mg lipid mixture. On the day of usage, the dose is be reconstituted by adding 50 ml of normal saline and mechanical shaking. This is followed by transfer to i.v. infusion bags and delivery to the patient's room.

The first cycle of platinum drug by the present invention was administered 7 days after a baseline thoracoscopy. At the time of thoracoscopy, an indwelling percutaneous catheter was placed in the chest wall for the intrapleural L-NDDP therapy. Before L-NDDP was administered into the pleural space through the catheter, approximately the same amount of pleural fluid was withdrawn immediately before drug administration. The drug concentration as administered was 2 mg NDDP/ml saline. The starting fluid volume infused was 225 ml. Dosages of up to about 450 mg/M² are useful, but the empirical end point is compromised respiration such as shortness of breath. The dose of L-NDDP was administered into the pleural space through the inserted catheter seven days after the date of thoracoscopy.

Repeated administrations were given every 3 to 4 weeks with attention to the presence or absence of a draining pleural effusion without loculation or (adhesions). That is a loculation surrounding the catheter entry must be broken u or carefully drained at the time of additional drug introduction to avoid hydrostatic pressure increases. Prior to infusion of L-NDDP, as much pleural fluid as possible will be removed and at least an amount of pleural fluid equal to the volume of L-NDDP to be infused. In a preferred regimen, before a third intrapleural administration of L-NDDP, a thoracoscopy was performed to provide photo-documentation of the tumor and tissue biopsy to assess the pathological tumor response. If the fluid was still draining through the indwelling catheter, the patient received a third treatment seven days after the this thoracoscopy. Patients treated by this protocol avoided extravasation and sepsis.

As a point of comparison, two of eight patients with similar presenting conditions were administered identical initial doses of L-NDDP, but via needle introduction to the pleural space with a collapsed lung. Upon lung reinflation, extravasation occurred, as the hydrostatic pressure rose and a needle hole was available for extravasation causing substantial deterioration in patient condition.

In some embodiments, doses were 450 mg NDDP/M² (2,250 mg lipid/m²) every 3 weeks, but about 150 mg NDDP/m² to about 1500 mg NDDP/m² are useful. Also contemplated is dosing from about every 5 days to about every other month.

Convenient infusion times include about 5 to about 60 minutes for all dose levels with particular reference to about 10 minutes. In some embodiments, it is useful to also administer i.v. 1 mg granisetron hydrochloride (Kytril™, SmithKline Beecham) or oral antiemetics at the completion of infusion.

While determining dose levels is a function of the disease stage and specifics of a patients condition, the accompanying toxicity scale is useful

TABLE 1

| Dose Level | NDDP Dose | Lipid Dose | Volume |
|---|---|---|---|
| −2 | 250 mg/m² | 3750 mg/m² | 125 ml/m² |
| −1 | 350 mg/m² | 5250 mg/m² | 175 ml/m² |
| 0 | 450 mg/m² | 6750 mg/m² | 225 ml/m² |
| +1 | 550 mg/m² | 8250 mg/m² | 275 ml/m² |

In some instances dose are usefully modified in subsequent infusions according to the following schedule:

| Granulocyte Nadir | Platelet Nadir | Modification |
|---|---|---|
| >1,000/mm³ | and >100,000/mm³ | Increase one level |
| 500-1,000/mm³ | and/or 50,000-100,000/mm³ | No change |
| <500/mm³ | and/or <50,000/mm³ | Decrease one level |
| Infection or | | Decrease one level |
| bleeding | | |
| Grade 0-1 | Increase one level | |
| Grade 2 | No change | |
| Grade 3-4 | Decrease one level or stop | |
| | treatment | |

This study comprised treatment of pleural disease by administration of intrapleural L-NDDP to patients with malignant pleural mesothelioma. While the course of treatment is within the skill of the medical professional with a view to the presenting condition of each patient, some initial parameters are noted. Intrapleural infusion is usefully held between consecutive cycles until the absolute granulocyte count is >1,500/mm³ and platelets >100,000/mm³ and complete recovery of non-hematological toxicities. Patients experiencing a delay in subsequent infusions of >2 weeks are advised to have a one dose level reduction.

### Example 1

A 47 year old male presented with left pleural effusion. He had a history of heavy smoking and prior asbestos exposure. Peural fluid was sampled. A CT scan of the chest disclosed minimal pleural thickening on the left side without lung parenchymal lesions or hilar or mediastinal adenopathy. Pleural fluid cytology was positive for malignant cells. The pathological diagnosis was malignant pleural mesothelioma. The patient was started on therapeutic doses of cisplatin, adriamycin and cyclophosphamide intravenously. The patient failed to respond to these drugs as determined by the amount of pleural fluid after 2 cycles of therapy. A thoracoscopy was performed which disclosed multiple plaque-like lesions in both pleuras. A transthoracic Denver catheter was left in situ entering in the pleural space. One week post-placement of the catheter, L-NDDP was introduced into the pleura through the catheter after draining an equivalent amount of pleural fluid, here 450 mg/m² and 225 mI/M². Administration occurred without extravasation.

The dosage was well tolerated with only a one time fever and chills about 4 hours after completion of the infusion.

A chest X-ray taken 3 weeks post-initial administration disclosed partial loculation of pleural fluid. A second dose of L-NDDP was administered at 3 weeks post-initial administration was preceded by the removal of an equivalent amount of pleural fluid.

At 6 weeks post-initial administration complete fluid loculation was observed, which prevented the administration of a third dosage of L-NDDP.

Over the next few months, fluid loculation completely disappeared. Chest X-rays performed at one year after completion of dosing showed a very small scar on the left lung basis. The subject has remained disease free for four years. Thereafter the patient re-presented with a local recurrence in the form of a chest wall mass without pleural infusion. The patient is alive at over 6 years post-treatment.

Particular note is made of the following publications:

1. Selikoff J J, Chung J, Hammond E C. “Relation between exposure to asbestos and mesothelioma.” N Eng J Med 272:560-565, 1965

2. Mossman B T, Bignon J, Corn M, Seaton A, Gee J B. “Asbestos: Scientific developments and implications for public policy.” Science 247:294-301, 1 990

3. Rogers A J, Leigh J, Berry G, Ferguson D A, Mulder H B, Ackad M. “Relationship between lung asbestos fiber type and concentration and relative risk of mesothelioma. A case control study.” Cancer 67:1912-1920, 1991

4. Borow M. Coston, A. Livornese L, Schalet N. “Mesothelioma following exposure to asbestos: A review of 72 cases.” Chest 64:646, 1973

5. Antman, K H. “Clinical presentation and natural history of benign and malignant mesothelioma.” Semin Oncol 8:313, 1981

6. Rusch V W, Piantadose S, Holmes E C. “The role of extrapleural pneumonectomy in malignant pleural mesothelioma. A Lung Cancer Study Group Trial.” J Thorac Cardiovasc Surg 102:1-9, 1991

7. Ginsberg R J. “Diffuse malignant mesothelioma: A therapeutic dilemma.” Ann Thorac Surg 42:608-612, 1986

8.Probst G, Buelzebruck H, Bauer H, Branscheld H G, Vogt-Moykopf J. “The role of pleuropeumonectomy in the treatment of diffuse malignant mesothelioma of the pleura.” Thoracic Surgery:Surgical Management of Pleural Disease. Deslauriers J, Lacquet L K, eds., (St. Louis; C V Mosby, 1990, ), 344-350.

9. Ball D L. Cruickshank D G. “The treatment of malignant mesothelioma of the pleura: Review of a 5-year experience, with special reference to radiotherapy.” Am J Clin Oncol 13:4-9, 1990

10. Gordon W, Antman K, Greenberger J, Weichselbaum R, Chaffey J. “Radiation therapy in the management of patients with mesothelioma.” Int J Radiat Oncol Biol Phys 8:19-25, 1982

11. Albers A S, Falkson F, Goodhals L, Vorobiof D, Vander Merwe C. “Malignant pleural mesothelioma: A disease unaffected by current therapeutic measures.” J Clin Oncol 6:527-535, 1988

12. Aisner J, Wiernick P H. “Chemotherapy in the treatment of malignant mesothelioma.” Semin Oncol 8:335-343, 1981

13. Samson M, Baker L, Wasser L et al. “Randomized comparison of cyclophosphamide, OTIC and Adriamyan vs. Cyclophosphamide and Adriamyan in patients with advanced malignant mesothelioma: A Sarcoma Intergroup study.” Proc Am Soc Clin Oncol 4:128, 1985 (Abstract)

14. Samson M, Banker L, Benjamin R S, Lane M, Plager C. “Cis-dichlorodiamineplatinum III in advanced soft tissue and bony sarcomas: A Southwest Oncology Group Study.” Cancer Treat Rep 63:2027-9, 1979

15. Raghavan D, Gianoutsosp, Bishop J et al. “Phase II trial of carboplatin in the management of malignant mesothelioma.” J Clin Oncol 8:151-154, 1990

16. Shin D M, Fossella F V, Umsawasdi T, et al. “Prospective study of combination chemotherapy with cyclophosphamide, doxorubicin, and cisplatin for unresectable or metastatic malignant pleural mesothelioma.” Cancer 1;76(1 1):2230-6, (1995).

17. Rusch V, Saltz L, Venkatraman E, et al. “A phase 11 trial of pleurectomy/decortication followed by intrapleural and systemic chemotherapy for malignant pleural mesothelioma.” J Clin Oncol 12:1156-1163, 1994

18. Walsh G L, Shin D M, Komaki R, McMurtrey M J, Hong W K, Roth J. “An aggressive multimodality protocol does not improve survival in patients with malignant pleural mesothelioma.” Presented at the 1994 Annual Meeting of the Society of Surgical Oncology, Houston Tex., March 17-20, 1994; 36 (Abstract)

19. Perez-Soler R, A K Knokhar, G Lopez-Berestein. “Treatment and prophylaxis of experimental liver metastasises of M5076 reticulosarcoma with cis-bis-neodecanoato-1,2-trans-R,R-diaminocyclohexane platinum (II) entrapped in multilamellar vesicles.” Cancer Res 47:6462-6466, 1987

20. Perez-Soler R, A K Khokhar, J Lautersztain, Mitchell P A, Smith K L. “Ultrastructural and freeze-fracture localization of multilamellar liposomes containing a lipophilic cisplatin analogue in normal tissues and liver metastasises or M5076 reticulosarcoma.” Cancer Drug Deliv. 1987;4(2):75-88

21. Lautersztain, J, R Perez-Soler, J Turpin, A R Khokhar, Z H Siddik, K Schmidt, G Lopez-Berestein. Cellular pharmacology of liposome entrapped cis-bis-neodecanoato-trans-R,R-1,2-diaminocyclohexane platinum (II). Cancer Res 48:1300-1306, 1988

22. Perez-Soler R, L Y Yang, B Drewinko, J Lautersztain, A R Khokhar. “Increased cytotoxicity and reversal of resistance to cisplatin with entrapment of cis-bis-neodecanoato-1,2-trans-R,R-diaminocyclohexane platinum (II) in multilamellar lipid vesicles.” Cancer Res 48:4509-4512, 1988

23. Khokhar A R, Wright K, Z H Siddik, R Perez-Soler. “Organ distribution and tumor uptake of liposome-entrapped cis-bis-neodecanoato-trans-R,R-1,2-diaminocyclo-hexane platinum (II) administered intravenously and into the proper hepatic artery.” Cancer Chemother Pharmacol 22:223-227, 1988

24. Khokhar A R, S Al-Baker, I H Krakoff, R Perez-Soler. “Toxicity and antitumor activity of cis-bis-carboxylato (trans-R,R-1,2-diaminocyclohexane) platinum (II) complexes entrapped in liposomes, a new series of lipid-soluble drugs.” Cancer Chemother Pharmacol. 23:219-224, 1989

25. Perez-Soler R, J Lautersztain, L Clifton Stephens, K Wright, A R Khokhar. “Pharmacology and toxicity of liposome-entrapped cis-bis-neodecanoato-trans-R,R-1,2-diaminocyclohexane platinum (II) in mice and dogs.” Cancer Chemother Pharmacol. 24:1-8, 1989

26. Perez-Soler R, A R Khokhar, J Lautersztain, S Al-Baker, K Francis, D Macias-Kiger, G Lopez-Berestein. “Clinical development of liposomal-platinum.” J Liposome Res 1 (4) 447-459, 1990

27. Perez-Soler R, G Lopez-Berestein, J Lautersztain, S Al-Baker, K Francis, D Macias-Kiger, M N Raber, A R Khokhar. “Phase I clinical and pharmacology study of liposome-entrapped cis-bis-neodecanoato-trans-R,R-1,2-diaminocyclohexane-platinum (II).” Cancer Res. 50:4254-4259,1990

28. Vadiei K, Siddik Z H, Khokhar A R, Al-Baker S, Sampedro F, Perez-Soler R. “Pharmacokinetics of liposome-entrapped cis-bis-neodecanoato-trans-R,R-1,2-diaminocyclohexane platinum (II) and cisplatin administered iv and ip in the rat.” Cancer Chemother Pharmacol 30:365-369, 1992

29. Andrews PA, S Velury, SC Mann, SB Howell. “Cis-diammenedichloroplatinum (II) accumulation in sensitive and resistant human ovarian carcinoma cells.” Cancer Cells 2:36-43, 1990

30. Han I, Ling Y H, Al-Baker S, Khokhar A R, Perez-Soler R. “Cellular pharmacology of liposomal-cis-bis-neodecanoato-trans-R,R-1,2-diaminocyclohexane platinum (II) (L-NDDP) and cisplatin in A2780 and A2780/PDD cells.” Cancer Res 53:4913-4919, 1993

31. Perez-Soler R, K Francis, S Al-Baker, et al. “Preparation and characterization of a liposomal preparation containing a lipophilic cisplatin derivative for clinical use.” J Microencapsulation 11:41-54, 1994

The teachings of the all publications cited throughout this disclosure are incorporated herein by reference herein.

I claim

1. A sepsis-avoiding method of administering platinum therapeutic agents into a body cavity of a subject by means of introduction through a hydrostatic-tight indwelling catheter.

2. A method of administering platinum therapeutic agents into a body cavity of a subject by the step of hydrostatic-neutral introduction.

3. The method of claim 1 further comprising hydrostatic-neutral introduction.

4. The method of claim 1 wherein said platinum therapeutic agent is selected from the group comprising NDDP, well as cisplatin (and derivatives such as polyamidoamine (PAMAM) dendrimer generation 3.5 with a sodium carboxylate surface conjugated to cisplatin giving a dendrimer-platinate (dendrimer-Pt; 20-25 wt % platinum), nedaplatin, JM335 (trans-ammine (cyclohexylaminedichlorodihydroxo) platinum(IV)), and its platinum(II) dichloro homolog JM334 (including their cis isomeric counterparts (JM 149 for JM335 and JM118 for JM334), JM216, ZD0473 (cis-amminedichloro(2-methylpyridine) platinum(II)); carboplatin, oxaliplatin, iproplatin; the dinuclear platinum complexes, BBR3005 ([trans-PtCl(NH3)22H2N(CH2)6NH2]2 +), BBR3171 ([cis-PtCl(NH3)22H2N(CH2)6NH2]2 +) and the trinuclear platinum complex, BBR3464 ([trans-PtCl(NH3)22 mu-trans-Pt(NH3)2(H2N(CH2)6NH2)2]4+); sterically hindered platinum complex, and AMD473 [cis-aminedichloro(2-methylpyridine) platinum (II)].

5. The method of claim 4 wherein said platinum therapeutic agent is in liposomal form.

6. The method of claim 5 wherein said platinum therapeutic agent is L-NDDP.
